(19) United States
(12) Patent Application Publication
(10) Pub. No.: US 2009/0320356 A1
(43) Pub. Date: Dec. 31, 2009

(54) STOCKPILING METHANOL AND/OR DIMETHYL ETHER FOR FUEL AND ENERGY RESERVES

(76) Inventors: George A. Olah, Beverly Hills, CA (US); G.K. Surya Prakash, Hacienda Heights, CA (US)

Correspondence Address: WINSTON & STRAWN LLP;PATENT DEPARTMENT, 1700 K STREET, N.W., WASHINGTON, DC, 20006, US

(21) Appl. No.: 12/469,366

(22) Filed: May 20, 2009

Related U.S. Application Data
(60) Provisional application No. 61/075,994, filed on Jun. 26, 2008.

(51) Int. Cl. C10L 1/18; C07C 43/04; C07C 31/04; C07C 1/20

(52) U.S. Cl. 44/448; 44/451; 568/671; 568/840; 585/639

(57) ABSTRACT

The present invention relates to a method of stockpiling a fuel source by storing methanol or dimethyl ether in appropriate storage facilities to provide an alternative fuel source that can be used to avoid shortages due to unavailability, limited availability or excessive costs of oil.

This application claims the benefit of application No. 61/075,994 filed Jun. 26, 2008, the entire content of which is expressly incorporated herein by reference thereto.

### FIELD OF THE INVENTION

The invention relates to methods of stockpiling alternative fuel sources by storing methanol and/or dimethyl ether to provide an alternative fuel source, wherein the stored methanol and/or dimethyl ether can be produced by chemical recycling of naturally existing, readily available compounds such as carbon dioxide and methane.

### BACKGROUND OF THE INVENTION

Hydrocarbons are essential in modern life. Hydrocarbons are used as fuel and raw material in various fields, including the chemical, petrochemical, plastics, and rubber industries. Fossil fuels, such as coal, oil and gas, are composed of hydrocarbons with varying ratios of carbon and hydrogen, and are non-renewably used when combusted, forming carbon dioxide and water. Despite their wide application and high demand, fossil fuels present a number of disadvantages, including the finite reserve, irreversible combustion and contribution to air pollution and global warming. Considering these disadvantages, and the increasing demand for energy, alternative sources of energy are needed.

Methanol, CH₃OH, is the simplest liquid oxygenated hydrocarbon, differing from methane (CH₄) by a single additional oxygen atom. Methanol, also called methyl alcohol or wood alcohol, is a colorless, water-soluble liquid with a mild alcoholic odor, and is easy to store and transport. It freezes at −97.6° C., boils at 64.6° C., and has a density of 0.791 at 20° C.

Methanol is not only a convenient and safe way to store energy, but is also an excellent fuel. Methanol can be blended with gasoline or diesel and used as fuels, for example in internal combustion engines or electricity generators. One of the most efficient uses of methanol is in fuel cells, particularly in direct methanol fuel cell (DMFC), in which methanol is directly oxidized with air to carbon dioxide and water while producing electricity.

Contrary to gasoline, which is a complex mixture of many different hydrocarbons and additives, methanol is a single chemical compound. It contains about half the energy density of gasoline, meaning that two liters of methanol provides the same energy as a liter of gasoline. Even though methanol's energy content is lower, it has a higher octane rating of 100 (average of the research octane number (RON) of 107 and motor octane number (MON) of 92), which means that the fuel/air mixture can be compressed to a smaller volume before being ignited. This allows the engine to run at a higher compression ratio (10-11 to 1 against 8-9 to 1 of a gasoline engine), more efficiently than a gasoline-powered engine. Efficiency is also increased by methanol's higher “flame speed,” which enables faster, more complete fuel combustion in the engines. These factors explain the high efficiency of methanol despite its lower energy density than gasoline. Further, to render methanol more ignitable even under the most frigid conditions, methanol can be mixed with gasoline, with volatile compounds (e.g., dimethyl ether), with other components or with a device to vaporize or atomize methanol. For example, an automotive fuel can be prepared by adding methanol to gasoline with the fuel having a minimum gasoline content of at least 15% by volume (M85 fuel) so that it can readily start even in low temperature environments. Of course, any replacement of gasoline in such fuels will conserve oil resources, and the amount of methanol to add can be determined depending upon the specific engine design.

Methanol has a latent heat of vaporization of about 3.7 times higher than gasoline, and can absorb a significantly larger amount of heat when passing from liquid to gas state. This helps remove heat away from the engine and enables the use of an air-cooled radiator instead of a heavier water-cooled system. Thus, compared to a gasoline-powered car, a methanol-powered engine provides a smaller, lighter engine block, reduced cooling requirements, and better acceleration and mileage capabilities. Methanol is also more environment-friendly than gasoline, and produces low overall emissions of air pollutants such as hydrocarbons, NOₓ, SO₂ and particulates.

Methanol is also one of the safest fuels available. Compared to gasoline, methanol's physical and chemical properties significantly reduce the risk of fire. Methanol has lower volatility, and methanol vapor must be four times more concentrated than gasoline for ignition to occur. Even when ignited, methanol burns about four times slower than gasoline, releases heat only at one-eighth the rate of gasoline fire, and is far less likely to spread to surrounding ignitable materials because of the low radiant heat output. It has been estimated by the EPA that switching from gasoline to methanol would reduce incidence of fuel-related fire by 90%. Methanol burns with a colorless flame, but additives can solve this problem.

Methanol also provides an attractive and more environment-friendly alternative to diesel fuel. Methanol does not produce smoke, soot, or particulates when combusted, in contrast to diesel fuel, which generally produces polluting particles during combustion. Methanol also produces very low emissions of NOₓ because it burns at a lower temperature than diesel. Furthermore, methanol has a significantly higher vapor pressure compared to diesel fuel, and the higher volatility allows easy start even in cold weather, without producing white smoke typical of cold start with a conventional diesel engine. If desired, additives or ignition improvers, such as octyl nitrate, tetrahydrofurfuryl nitrate, peroxides or higher alkyl ethers, can be added to bring methanol's cetane rating to the level closer to diesel. Methanol can also be used in the manufacture of biodiesel fuels by esterification of fatty acids.

Closely related and derived from methanol, and also a desirable alternative fuel is dimethyl ether. Dimethyl ether is easily obtained from methanol by dehydration. Dimethyl ether or CH₃OCH₃, the simplest of all ethers, is a colorless, nontoxic, non-corrosive, non-carcinogenic and environmentally friendly chemical that is mainly used today as an aerosol propellant in spray cans, in place of the banned CFC gases. Dimethyl ether has a boiling point of −25° C., and is a gas under ambient conditions. Dimethyl ether is, however, easily handled as liquid and stored in pressurized tanks, much like liquefied petroleum gas (LPG). The interest in dimethyl ether as alternative fuel lies in its high cetane rating of 55 to 60, which is much higher than that of methanol and is also higher than the cetane rating of 40 to 55 of conventional diesel fuels. The cetane rating indicates that dimethyl ether can be effectively used in diesel engines. Advantageously, dimethyl ether, like methanol, is clean burning, and produces no soot particulates, black smoke or SO₂, and only very low amounts of NOₓ and other emissions even without after-treatment of its exhaust gas.

Another methanol derivative is dimethyl carbonate, which can be obtained by converting methanol with phosgene or by oxidative carbonylation of the methanol. Dimethyl carbonate has a high cetane rating, and can be blended into diesel fuel in a concentration up to 10%, reducing fuel viscosity and improving emissions.

Methanol and its derivatives, e.g., dimethyl ether, dimethyl carbonate, and biodiesel fuel, have many existing and potential uses. They can be used, for example, as a substitute for gasoline and diesel fuel in ICE-powered cars with only minor modifications to the existing engines and fuel systems. Methanol can also be used in fuel cells, for fuel cell vehicles (FCVs), which are considered to be the best alternative to ICEs in the transportation field. Dimethyl ether is also a potential substitute for LNG and LPG for heating homes and in industrial uses.

Methanol is also an attractive source of fuel for static applications. For example, methanol can be used directly as fuel in gas turbines to generate electric power. Gas turbines typically use natural gas or light petroleum distillate fractions as fuel. Compared to such fuels, methanol can achieve higher power output and lower NOₓ emissions because of its lower flame temperature. Since methanol does not contain sulfur, SO₂ emissions are also eliminated. Operation on methanol offers the same flexibility as on natural gas and distillate fuels, and can be performed with existing turbines, originally designed for natural gas or other fossil fuels, after relatively easy modification. Methanol is also an attractive fuel since fuel-grade methanol, with lower production cost than higher purity chemical-grade methanol, can be used in turbines.

The present inventors have discovered improved and novel methods of production for methanol and/or dimethyl ether, using the hydrogenative chemical recycling of carbon dioxide. These methods are disclosed in published U.S. Patent Application Nos. 2006/0235088, 2006/0235091, and 2007/0254969, as well as issued U.S. Pat. Nos. 5,928,806 and 7,378,561, the entire content of which are expressly incorporated herein by reference thereto. These methods enable the use of methanol and dimethyl ether as renewable energy sources, offering an environmentally neutral carbon dioxide balance for the use of these efficient fuels and their derived synthetic hydrocarbon products, while mitigating the effect of carbon dioxide as a greenhouse gas on our global climate.

At present, the world is facing an oil crisis, caused by rapid depletion of natural resources and our increasing use of technology that requires fuel. National oil reserves presently provide a cushion for major oil or natural gas emergencies and help to avoid disastrous disruptions caused by natural causes, as well as by geopolitical or economic interruption of these sources.

The United States government has recognized this crisis; the Strategic Petroleum Reserve (SPR) was established in the 1970s to maintain an emergency oil supply, and the Energy Policy Act of 2005 directed the Secretary of Energy to fill the SPR to its 1 billion barrel capacity. Unfortunately, there have been several challenges to meeting this directive, including emergency situations like Hurricane Katrina, the on-going turbulence in the middle-east, and the overall oil shortage. Furthermore, storage of oil, by its nature, poses several safety issues, for example, its extreme flammability.

Much concern is expressed for independence from imported sources, and efforts are ongoing to find alternative sources of domestic energy and fuel sources. In addition to concerns regarding energy sources, there are additional concerns related to storage and transportation. Regardless of how energy is generated, its storage and transport is a difficult, technologically complex and expensive problem for which no easily applicable, safe and economic solutions are yet available. The United States government has recognized all these challenges, as evidenced by Section 369(h) of the Energy Policy Act of 2005, creating a task force on Strategic Unconventional Fuels to “accelerate the development of unconventional fuels”.

One of the presently considered ways to store energy is in the form of hydrogen. Hydrogen is however a highly volatile and explosive gas, which is not present in its free form on earth because of its high reactivity with oxygen (an essential major constituent of our atmosphere). It must be consequently generated from its compounds such as water (by electrolysis or thermal high energy conversion) or produced from hydrocarbons. Storage of any significant amount of hydrogen necessitates costly cryoscopic and high-pressure conditions for its liquification. Its extreme light nature and high volatility further causes ready diffusion through most materials and increased explosion danger. Consequently, hydrogen is not suited for establishing large-scale feasible strategic storage facilities or readily transportation and commercial use.

The present invention provides a way to meet the need for establishing fuel reserves, particularly by providing a reserve of unconventional fuel that be safely stored and transported, without being subject to the issues posed by obtaining and storing oil or other existing alternative fuel sources.

### SUMMARY OF THE INVENTION

One embodiment of the invention relates to a method of stockpiling a fuel source by storing methanol or dimethyl ether in appropriate storage facilities in an amount sufficient to provide an alternative fuel source that can be used to avoid shortages due to unavailability, limited availability or excessive costs of oil. The storage facility can be a natural or man-made storage facility providing safe, economic, and convenient storage of the methanol or dimethyl ether, such as an underground tank, an above-ground tank, or a salt dome. Stockpiling can be conducted in a manner to help achieve the goals of developing unconventional fuels as set forth in Section 369(h) of the United States Energy Policy Act of 2005.

Stored methanol can be made by reductive conversion of carbon dioxide obtained from one or more of (a) an exhaust stream from a fossil fuel burning power or industrial plant, (b) a source accompanying natural gas, or (c) the atmosphere with the carbon dioxide obtained by absorbing atmospheric carbon dioxide onto a suitable adsorbent followed by treating the adsorbent to release the adsorbed carbon dioxide therefrom. The obtained carbon dioxide can be reduced under conditions sufficient to produce a reaction mixture that contains formic acid and formaldehyde, methanol and methane, followed, without separation of the reaction mixture, by a treatment step conducted under conditions sufficient to convert the formaldehyde to formic acid and methanol.

The obtained carbon dioxide can be reduced to form carbon monoxide, which is then reacted with methanol under conditions sufficient to obtain methyl formate, and the methyl formate is catalytically hydrogenated under conditions sufficient to produce methanol. The hydrogen needed for the hydrogenation of methyl formate can be obtained by decomposing at least some of the formic acid from the reaction mixture; by reacting carbon dioxide with methane, natural gas or carbon dioxide; or by electrolysis or catalytic or thermal cleavage of water.

Alternatively, the stored methanol and can be made by combining wet reforming and dry reforming of sufficient amounts of methane, carbon dioxide and water under reaction conditions sufficient to produce a mixture of carbon monoxide and hydrogen; and converting the carbon monoxide and hydrogen of the mixture under conditions sufficient to form methanol. The combined wet and dry reforming can be conducted in single or multiple steps at a temperature of about 800 to 1100° C. in the presence of a metal or metal oxide catalyst. In another embodiment of the invention, stored dimethyl ether can be prepared by dehydrating methanol under conditions sufficient to produce dimethyl ether for storage.

An additional embodiment relates to a method of preventing a fuel shortage due to unavailability or excessive cost of oil, by stockpiling methanol or dimethyl ether in appropriate storage facilities as disclosed herein; retrieving the methanol or dimethyl ether from the storage facilities; and preparing an alternative fuel from the methanol or dimethyl ether in an amount sufficient to at least partially counteract the fuel shortage.

The stored and retrieved dimethyl ether can be used as a substitute for natural gas or LPG; is mixed with conventional diesel fuel to form an improved diesel fuel; or is converted to ethylene, propylene, higher olefins, synthetic hydrocarbons or aromatics for use as fuels, fuel supplements or fuel additives. The stored, retrieved dimethyl ether can be converted to ethylene or propylene, which in turn is hydrated to form ethanol or propanol for use as fuels, fuel supplements or fuel additives. In a further embodiment, stored, retrieved methanol can be added to gasoline to form an alternative fuel having a minimum gasoline content of at least 15% by volume.

An additional embodiment of the invention relates to a method of reducing U.S. dependency on foreign oil, which method includes stockpiling methanol or dimethyl ether in appropriate storage facilities as disclosed herein; retrieving the methanol or dimethyl ether from the storage facilities; and preparing an alternative fuel from the methanol or dimethyl ether in an amount sufficient to reduce dependency on foreign oil. Stored and retrieved dimethyl ether can be used either as a substitute for natural gas or LPG; can be mixed with conventional diesel fuel to form an improved diesel fuel; or can be converted to ethylene, propylene, higher olefins, synthetic hydrocarbons or aromatics for use as fuels, fuel supplements or fuel additives. Alternatively, the stored and retrieved dimethyl ether can be converted to ethylene or propylene, which in turn is hydrated to form ethanol or propanol for use as fuels, fuel supplements or fuel additives. Stored and retrieved methanol can be added to gasoline to form an alternative fuel having a minimum gasoline content of at least 15% by volume.

### DETAILED DESCRIPTION OF THE PREFERRED EMBODIMENTS

The present invention provides for convenient storage of methanol and/or dimethyl ether as strategic reserve fuels that can be readily and effectively stored in natural or man-made storage facilities from which they can be readily withdrawn for use. As methanol and dimethyl ether can essentially be produced from recycling CO₂ from any sources, including the air, with hydrogen provided by water and utilizing any energy source, the present method of stockpiling of fuel and energy reserves in the form of methanol and/or dimethyl ether provides a convenient new way for safeguarding against energy and fuel emergencies and shortages.

Stockpiling of methanol offers several advantages over stockpiling oil. First, methanol is far less flammable than oil and other hydrocarbons, having a boiling point of 64.6° C. (54° F.) at atmospheric pressure. Gasoline, in contrast, will ignite at temperatures below freezing. Also, methanol is naturally present and found essentially non-toxic in plant and animal studies. For humans, methanol is safe at low concentrations. As a result of methanol's ready availability and relative safety, the storage thereof is far less expensive than oil and other fuels. Due to its physical properties, methanol is also easy to transport.

Dimethyl ether can also be conveniently stored and handled in the same manner as liquefied petroleum gas. Dimethyl ether is a gas at room temperature, so that it is pressurized to a liquid to facilitate handling. It generally should be stored in pressurized tanks or similar vessels.

Both methanol and dimethyl ether can be conveniently stored for stockpiling reserves in natural geological formations or man-made facilities under conditions requiring only limited, if any, alteration of existing oil or natural gas storage facilities. For example, methanol and dimethyl ether can be stored in above-ground or underground tanks, similar to those used for gasoline or ethanol storage. Thus, existing facilities, previously built for the purposes of storing more conventional fuels such as oil, can be easily adapted to store methanol and dimethyl ether, without incurring significant extra cost. Additionally, methanol and dimethyl ether can be stored in natural facilities, such as natural subterranean cavities, salt domes, exhausted mine shafts, and oil fields. To be stored as a liquid, dimethyl ether needs to be pressurized whereas methanol does not require pressurization as it already is a liquid at room temperature.

Methanol is a suitable liquid fuel which can be readily and efficiently produced from natural gas or coal, or by the methods as set forth in published U.S. Patent Application Nos. 2008/0039538, 2007/0254969, 2006/0235091, and 2007/0254969, as well as issued U.S. Pat. Nos. 5,928,806 and 7,378,561, all of which are incorporated by reference. For example, methanol can be produced by conversion of carbon dioxide by its bi-reforming with methane, or its reductive catalytic hydrogenation or electrochemical reduction with water. The inventors' BIREFORMING™ method includes reacting methane under a specific combination of conditions of wet (steam) and dry (CO₂) reforming with a specific mole ratio of reactants sufficient to produce a syn-gas mixture of carbon monoxide and hydrogen (CO/H₂) in a ratio of about 1:2, with a further reaction to convert the CO and H₂ to methanol, as shown by the following reaction:

3CₙH₍₂ₙ₊₂₎+(3n−1) H₂O+CO₂→(3n+1) CO+(6n+2) H₂

(n=1 representing methane itself)

Methanol can also be produced by biological means such as enzymatic conversions of varied biomaterials, such as biogas, which is produced in the digestive tracks of most mammals and other organisms, such as termites and bacteria. Dimethyl ether is a derived product of methanol produced by dehydration of methanol, or by the BIREFORMING™ method mentioned herein.

Additionally, methanol and dimethyl ether can be made by the reduction of carbon dioxide obtained from exhaust produced by various sources, including exhaust from fuel burning sources (e.g., industrial plants), or the atmosphere, as set forth in published U.S. Patent Application Nos. 2008/0039538 and 2007/0254969, and U.S. Pat. No. 7,378,561. This can also include the BIREFORMING™ method for carbon dioxide, or its reductive catalytic hydrogenation or electrochemical reduction in water. In yet another embodiment of the invention, the reduction of carbon dioxide occurs under conditions that result in a mixture of formic acid and formaldehyde, methanol, and methane, followed by conversion of formaldehyde to formic acid and methanol, as set forth in published U.S. Patent Application Nos. 2008/0039538 and 2007/0254969.

As set forth in published U.S. Patent Application No. 2008/0039538, chemical feedstocks can be prepared from methanol and dimethyl ether, such as ethylene and propylene, higher olefins, synthetic hydrocarbons, or aromatics. Ethylene and propylene can be then be hydrated to form ethanol or propanol. Methanol can be added to gasoline for use as an alternative fuel, known as M85, and dimethyl ether can also be used a substitute for natural gas or liquid petroleum gas (LPG). There are several applications for methanol and dimethyl ether as fuels; these are just a few of the examples.

As shown herein, methanol and dimethyl ether are versatile, economic, environmentally friendly, and readily available sources of fuel. Not only do they offer a desirable way to replenish diminishing fuel reserves, they also offer convenient storage for energy generated in a variety of ways. The ability to store and stockpile energy conveniently in the form of methanol and/or dimethyl ether fuels is of substantial significance as it involves the use of renewable, environmentally carbon neutral fuels. Needed energy for their production can come from any energy source, including off-peak fossil fuel burning plants, atomic power plants, or any alternate energy source based on solar, hydro, wind or the wave.

With time, it is expected that the availability of fossil fuels will continue to decrease, while their demand and cost will only increase. The need for alternative fuel sources has never been so apparent, with governments across the world taking steps to identify and utilize such fuels. The present invention meets this need, by providing a safe, economically feasible, and environmentally friendly way of stockpiling alternative fuel, potentially eliminating crisis situations that are caused by catastrophic events, human use, or simply fears of shortage.

What is claimed is:

1. A method of stockpiling a fuel source comprising storing methanol or dimethyl ether in appropriate storage facilities in an amount sufficient to provide an alternative fuel source that can be used to avoid shortages due to unavailability, limited availability or excessive costs of oil.

2. The method of claim 1, wherein the storage facility is a natural or man-made storage facility that provides safe, economic, and convenient storage of the methanol or dimethyl ether.

3. The method of claim 2, wherein the storage facility is an underground tank, an above-ground tank, or a salt dome.

4. The method of claim 1, wherein the stockpiling is conducted in a manner to help achieve the goals of developing unconventional fuels as set forth in Section 369(h) of the United States Energy Policy Act of 2005.

5. The method of claim 1, wherein methanol is stored, with the methanol being made by reductive conversion of carbon dioxide obtained from one or more of (a) an exhaust stream from a fossil fuel burning power or industrial plant, (b) a source accompanying natural gas, or (c) the atmosphere with the carbon dioxide obtained by absorbing atmospheric carbon dioxide onto a suitable adsorbent followed by treating the adsorbent to release the adsorbed carbon dioxide therefrom.

6. The method of claim 5 wherein the obtained carbon dioxide is reduced under conditions sufficient to produce a reaction mixture that contains formic acid and formaldehyde, methanol and methane, followed, without separation of the reaction mixture, by a treatment step conducted under conditions sufficient to convert the formaldehyde to formic acid and methanol.

7. The method of claim 5 wherein the obtained carbon dioxide is reduced to form carbon monoxide, the carbon monoxide is reacted with methanol under conditions sufficient to obtain methyl formate, and the methyl formate is catalytically hydrogenated under conditions sufficient to produce methanol.

8. The method of claim 7, wherein the hydrogen needed for the hydrogenation of methyl formate is obtained by decomposing at least some of the formic acid from the reaction mixture; by reacting carbon dioxide with methane, natural gas or carbon dioxide; or by electrolysis or catalytic or thermal cleavage of water.

9. The method of claim 1, wherein methanol is stored, with the methanol being made by combining wet reforming and dry reforming of sufficient amounts of methane, carbon dioxide and water under reaction conditions sufficient to produce a mixture of carbon monoxide and hydrogen; and converting the carbon monoxide and hydrogen of the mixture under conditions sufficient to form methanol.

10. The method of claim 9, wherein the combined wet and dry reforming is conducted in single or multiple steps at a temperature of about 800 to 1100° C. in the presence of a metal or metal oxide catalyst.

11. The method of claim 1, wherein dimethyl ether is stored, with the dimethyl ether prepared by reducing methanol under conditions sufficient to produce dimethyl ether for storage.

12. A method of preventing a fuel shortage due to unavailability or excessive cost of oil, which method comprises stockpiling the methanol or dimethyl ether in appropriate storage facilities according to the method of claim 1; retrieving the methanol or dimethyl ether from the storage facilities; and preparing an alternative fuel from the methanol or dimethyl ether in an amount sufficient to at least partially counteract the fuel shortage.

13. The method of claim 12, wherein dimethyl ether is stored and retrieved and then is either used as a substitute for natural gas or LPG; is mixed with conventional diesel fuel to form an improved diesel fuel; or is converted to ethylene, propylene, higher olefins, synthetic hydrocarbons or aromatics for use as fuels, fuel supplements or fuel additives.

14. The method of claim 12, wherein dimethyl ether is stored and retrieved and then converted to ethylene or propylene which in turn is hydrated to form ethanol or propanol for use as fuels, fuel supplements or fuel additives.

15. The method of claim 12, wherein methanol is stored and retrieved and then is added to gasoline to form an alternative fuel having a minimum gasoline content of at least 15% by volume.

16. A method of reducing U.S. dependency on foreign oil which method comprises stockpiling the methanol or dimethyl ether in appropriate storage facilities according to the method of claim 1; retrieving the methanol or dimethyl ether from the storage facilities; and preparing an alternative fuel from the methanol or dimethyl ether in an amount sufficient to reduce dependency on foreign oil.

17. The method of claim 16, wherein dimethyl ether is stored and retrieved and then is either used as a substitute for natural gas or LPG; is mixed with conventional diesel fuel to form an improved diesel fuel; or is converted to ethylene, propylene, higher olefins, synthetic hydrocarbons or aromatics for use as fuels, fuel supplements or fuel additives.

18. The method of claim 15, wherein dimethyl ether is stored and retrieved and then converted to ethylene or propylene which in turn is hydrated to form ethanol or propanol for use as fuels, fuel supplements or fuel additives.

19. The method of claim 15, wherein methanol is stored and retrieved and then is added to gasoline to form an alternative fuel having a minimum gasoline content of at least 15% by volume.
